(19) United States
(12) Patent Application Publication
(10) Pub. No.: US 2004/0126766 A1
(43) Pub. Date: Jul. 1, 2004

(54) Breakaway seal for processing a subarray of an array

(76) Inventor: Douglas A. Amorese, Los Altos, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/329,887

(22) Filed: Dec. 26, 2002

(51) Int. Cl. C12Q 1/68; G01N 33/53; G01N 33/542; C12M 1/34

(52) U.S. Cl. 435/6; 435/7.9; 435/287.2

(57) ABSTRACT

The present invention provides for assaying as few as a single microarray of a chemical array of microarrays at a time without contamination or damage to a remainder of the microarrays. A chemical array apparatus, system and method comprise a microarray attached to a surface of a planar substrate and a breakaway seal or barrier applied to the substrate surface to surround the microarray. The breakaway seal provides the microarray fluid isolation and is manipulatable. The system further comprises a cover extending over and in contact with the breakaway seal to cover the microarray. The method further comprises processing a microarray of the chemical array with a fluid, and breaking away a portion of the breakaway seal to create a gap. The gap releases the fluid in a direction away from remaining microarrays of the chemical array. A removable cover overlies a microarray on the chemical array apparatus.

### TECHNICAL FIELD

This invention relates to chemical arrays. In particular, the invention relates to independently processing individual microarrays of a chemical array.

### BACKGROUND ART

A variety of methods and materials are known and are currently available for fabricating arrays of chemical or biological materials, such as arrays of nucleic acids molecules or proteins. Common methods of making arrays involve in situ synthesis of chemical or biochemical polymers on predefined regions or features of an array substrate from sequential addition of monomer components that make up the polymers. Other common methods of making arrays involve immobilizing presynthesized polymers on an array substrate in an array format. Chemical and biochemical arrays (hereinafter ‘chemical arrays’) are useful for at least diagnostic, analytical, and research applications.

Chemical arrays enable researchers to analyze or screen a single test or target sample for a multitude (i.e., thousands) of characteristics very efficiently. Use of cDNA arrays for gene expression monitoring enable scientists to monitor changes in large numbers of genes (biological pathways) in a single experiment. However, in some applications there is a need to look at a few genes or pathways with larger number of test samples. This is accomplished using non-array methods, such as a Real Time-Polymerase Chain Reaction (RT-PCR) approach, a Taqman approach, or using what some skilled in the art refer to as an ‘array of arrays’ approach.

Advances in the fabrication technology and equipment has provided for a chemical array that comprises a plurality of microarrays or subarrays fabricated in a corresponding plurality of spatially defined locations or regions on a single array substrate or support. The ‘array of arrays’ approach typically uses a plurality of microarrays of identical chemical content on the array substrate. However, the chemical content of the plurality of microarrays need not be identical. Hereinafter, the chemical ‘array of arrays’ will be referred to as a chemical array of microarrays and the chemical content of the microarrays on the array substrate is the same or at least one of the microarrays is different from the remainder of the microarrays on the substrate. Typically, physical boundaries separate the plurality of microarrays from one another. For example, the wells or depressions of a microtiter plate physically separate and hold the plurality of microarrays. However, the depressions or wells in the microtiter plate tend to prevent direct use of the microtiter plate in various detection formats, such as calorimetric, fluorescent and radioactive detection formats, for which a flat surface is desired. Alternatively, the chemical arrays of microarrays are spatially separated on a substantially flat or planar support, such as a glass slide or polymer sheet. However, spatial separation does not address cross contamination during processing of individual microarrays with fluids.

In order to process a single microarray of the array of microarrays on a substantially flat solid support, typically a gasket is clamped in place to isolate the single microarray from the remainder of the microarrays on the substrate. The clamped gasket provides adequate fluid isolation, so that the processing of the single microarray does not contaminate the remaining microarrays. However, gaskets and clamps are cumbersome to work with and may involve repeated assembly and disassembly for processing the single microarray. Such repeated handling of the assembly increases the risk of damaging the remaining microarrays.

Further, a cover slip is placed over the single microarray during processing, such as when performing a hybridization of the single microarray with a target sample. The cover slip attempts to minimize evaporation of target sample during incubation among other things. This technique also makes screening and handling of the array of microarrays inconvenient and time intensive.

Thus, it would be advantageous to be able to perform an assay on individual microarrays of a chemical array of microarrays one at a time without contaminating or damaging a remainder of the microarrays of the array during each assay. Further, it would be desirable if such individual microarrays could be assayed separately in time without using cumbersome gaskets and clamps to handle and protect the chemical array. The ability to perform independent assays without contamination, damage and cumbersome handling of the chemical array would solve a longstanding need in the art.

### SUMMARY OF THE INVENTION

The present invention provides for processing as few as a single microarray of a chemical array of microarrays at a time separately from a remainder of microarrays of the array without contamination or damage to the remainder of microarrays and without using gaskets and clamps.

In one aspect of the invention, a chemical array apparatus is provided. The chemical array apparatus comprises a breakaway seal applied to a surface of a planar substrate. The breakaway seal surrounds a microarray attached to the substrate surface. A portion of the breakaway seal is removable to create a gap in the breakaway seal.

In another aspect of the invention, a system for processing a microarray of a chemical array is provided. The system comprises a chemical array that comprises a microarray attached to a surface of a planar substrate. The system further comprises a breakaway seal provided on the planar substrate to surround the microarray. A portion of the breakaway seal is removable to create a gap in the breakaway seal. The system still further comprises a removable cover extending over and in contact with the breakaway seal to shield the microarray.

In still another aspect of the invention, a method of assaying a microarray of a chemical array of microarrays is provided. The method comprises applying a breakaway seal to a surface of a planar substrate to ultimately surround a microarray on the planar substrate. The method further comprises processing the microarray with a fluid that is deposited on the microarray. The breakaway seal retains the fluid with the microarray. The method further comprises breaking away a portion of the breakaway seal that retains the fluid with the microarray. The broken away portion creates a gap in the breakaway seal. The gap provides an exit for the release of the fluid from the microarray.

In yet another aspect of the present invention, a removable cover for a chemical array apparatus is provided. The removable cover comprises a sheet of material that overlies a microarray of the chemical array apparatus. The sheet is removable to provide fluid access to the microarray.

One or more of the following advantages may be realized by using the present invention. The present invention allows for processing one microarray at a time on an array of microarrays. A single microarray can be assayed without contaminating the remaining microarrays on the array with the assaying solutions used to process the single microarray. The present invention protects the remaining microarrays during the processing of the single microarray from handling damage. The array can be stored until a remaining microarray thereon is used in a subsequent assay. Certain embodiments of the present invention have other advantages in addition to and in lieu of the advantages described hereinabove. These and other features and advantages of the invention are detailed below with reference to the following drawings.

### BRIEF DESCRIPTION OF THE DRAWINGS

The various features and advantages of the present invention may be more readily understood with reference to the following detailed description taken in conjunction with the accompanying drawings, where like reference numerals designate like structural elements, and in which:

FIG. 1 illustrates a surface or top view of a chemical array apparatus according to an embodiment of the present invention.

FIG. 2 illustrates a cross sectional view of the chemical array apparatus taken along line 2-2 in FIG. 1 according to an embodiment of the present invention.

FIG. 3 illustrates a magnified view of a portion of the chemical array apparatus in an encircled area labeled ‘3’ in FIG. 2 during an assay according to an embodiment of the present invention.

FIG. 4 illustrates a top or surface view of a chemical array apparatus according to another embodiment of the present invention.

FIG. 5 illustrates a surface or top view of the chemical array apparatus of FIG. 4 with a gap in the breakaway seal or barrier according to an embodiment of the present invention.

FIG. 6 illustrates a surface or top view of the chemical array apparatus of FIG. 4 with a gap in the breakaway seal or barrier according to another embodiment of the present invention.

FIG. 7 illustrates a top or surface view of an embodiment of a system for processing a microarray of a chemical array according to another aspect of the present invention.

FIG. 8 illustrates a cross sectional view of a system for processing a microarray of a chemical array according to another embodiment of the present invention.

FIG. 9 illustrates a perspective view of a system for processing a microarray of a chemical array according to another embodiment of the present invention.

FIG. 10 illustrates a side view of a system for processing a microarray of a chemical array according to another embodiment of the present invention.

FIG. 11 illustrates a side view of a system for processing a microarray of a chemical array according to another embodiment of the present invention.

FIG. 12 illustrates a flow chart of an embodiment of a method of assaying a microarray of a chemical array of microarrays according to another aspect of the present invention.

### DETAILED DESCRIPTION

Definitions

In the present application, unless a contrary intention appears, the following terms refer to the indicated characteristics. A “biopolymer” is a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another.

A “nucleotide” refers to a sub-unit of a nucleic acid and has a phosphate group, a five carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and the references cited therein (all of which are incorporated herein by reference), regardless of the source.

An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A biomonomer fluid or a biopolymer fluid refers to a liquid containing either a biomonomer or biopolymer, respectively (typically in solution). A “biochemical” refers to a biomonomer, a biomonomer fluid, an oligonucleotide, an oligonucleotide fluid, a biopolymer, a biopolymer fluid, or any reagent used in the fabrication of a biological array. A “chemical” refers to any and all chemical substances used in the fabrication of a chemical array, including biochemicals used in the fabrication of a biological array.

A “microarray”, unless a contrary intention appears, includes any one-, two- or three-dimensional arrangement of addressable features bearing a particular chemical moiety or moieties (for example, biopolymers such as polynucleotide sequences) associated with that region. A microarray is “addressable” in that it has multiple features of different moieties (for example, different polynucleotide sequences) such that a feature of the microarray at a particular predetermined location (an “address”) on the microarray will detect a particular target or class of targets (although a feature may incidentally detect non-targets of that feature). Microarray features are typically, but need not be, separated by intervening spaces.

An “array”, unless a contrary intention appears, includes any one-, two- or three-dimensional arrangement of addressable microarrays bearing chemical moieties (for example, biopolymers such as polynucleotide sequences) associated with that microarray. An array is “addressable” in that it has multiple microarrays of different moieties (for example, different polynucleotide sequences) such that a region including a microarray at a particular predetermined location (an “address”) on the array will detect a particular target or class of targets (although a feature may incidentally detect non-targets of that microarray). Array regions are typically discrete or separated by intervening spaces. In the case of an array or a microarray, the “target” will be referenced as a moiety in a mobile phase (typically fluid), to be detected by probes (“target probes”) which are bound to the substrate at the various regions and features. However, either of the “target” or “target probes” may be the one that is to be evaluated by the other (thus, either one could be an unknown mixture of polynucleotides to be evaluated by binding with the other).

A “microarray layout” refers to one or more characteristics of the features, such as feature positioning on the substrate within the microarray, one or more feature dimensions, and an indication of a moiety at a given location. An “array layout” refers to one or more characteristics of the regions of microarrays, such as region positioning on the substrate, one or more region dimensions, and an indication of a moiety or moieties in a given region.

“Hybridizing” and “binding”, with respect to polynucleotides, are used interchangeably. A “feature” refers to any finite small area on the microarray that can be illuminated and any resulting fluorescence therefrom simultaneously (or shortly thereafter) detected, for example a pixel. A “region” refers to any finite area on the array that includes a microarray. The microarray comprises the features that can be illuminated, as mentioned above.

An ‘array pattern’ refers to a spatially addressable or ordered arrangement of regions. A ‘subarray pattern’ refers to a spatially addressable or ordered arrangement of features. A ‘grid pattern’ refers to a spatially addressable or ordered arrangement of barriers or frame units surrounding the regions of the array pattern. A ‘scored pattern’ refers to a spatially ordered arrangement of scored lines or partial perforations in a cover material. A ‘zone’ refers to one of the regions surrounded by the seal or barrier and/or covered by the cover material.

A ‘seal or barrier’ refers to either a physical boundary or a chemical boundary created by the present invention, through which a fluid cannot flow, such that the seal or barrier retains a fluid within the boundaries of the seal or barrier. A ‘breakaway’ seal or barrier is when the seal or barrier is workable or manipulatable. By ‘workable’ or ‘manipulatable’, it is meant that a portion of the seal or barrier is removable such that a gap in the boundary of the seal or barrier is created. Fluid will flow through the gap. A ‘fluid’ is used herein to reference a liquid.

When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

Reference to a singular item, includes the possibility that there are plural of the same items present. “May” means optionally. The terms “upper” or “lower” are used in a relative sense only. Methods recited herein may be carried out in any order of the recited events, which is logically possible, as well as the recited order of events. All patents and other references cited in this application are incorporated into this application by reference except insofar as they may conflict with those of the present application (in which case the present application prevails).

Array Description

Any given substrate may carry one, two, three, four or more arrays or microarrays disposed on a front surface of the substrate. Depending upon the use, any or all of the arrays or microarrays may be the same or different from one another and each may contain multiple spots or features. A typical array or microarray may contain more than ten, more than one hundred, more than one thousand, more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments, each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features may be of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array or microarray may cover an area of less than 100 cm², or even less than 50 cm², 10 cm² or 1 cm². In many embodiments, the substrate carrying the one or more arrays or microarrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays or microarrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays and microarrays can be fabricated using drop deposition from pulse jets of either polynucleotide precursor units (such as monomers) in the case of in situ fabrication, or the previously obtained polynucleotide. Such methods are described in detail in, for example, the references including U.S. Pat. Nos. 6,242,266; 6,232,072; 6,180,351; 6,171,797; and 6,323,043; U.S. patent application Ser. No. 09/302,898, filed Apr. 30, 1999, by Caren et al.; and the references cited therein. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays or microarrays are made by photolithographic methods.

Reading Array Material

Following receipt by a user, an array made by an apparatus or a method of the present invention will typically be exposed to a sample (for example, a fluorescently labeled polynucleotide or protein containing sample) and the array then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications: Ser. No. 10/087,447, “Reading Dry Chemical Arrays Through The Substrate” by Corson et al.; and Ser. No. 09/846,125, “Reading Multi-Featured Arrays” by Dorsel et al. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. Nos. 6,251,685 and 6,221,583, and elsewhere). A result obtained from the reading may be used in that form or may be further processed to generate a result such as that obtained by forming conclusions based on the pattern read from the array (such as whether or not a particular target sequence may have been present in the sample, or whether or not a pattern indicates a particular condition of an organism from which the sample came). A result of the reading (whether further processed or not) may be forwarded (such as by communication) to a remote location if desired, and received there for further use (such as further processing).

### MODES FOR CARRYING OUT THE INVENTION

The present invention provides a chemical array apparatus, a system and a method wherein as few as one microarray of the chemical array apparatus is processable at a time separately from a remainder of microarrays of the chemical array apparatus. The processing of an individual or single microarray of the chemical array apparatus is performed separately in time from processing the remaining microarrays. Moreover, the processing is performed without contamination or damage to the remaining microarrays. Any one or more of the remaining microarrays of the chemical array apparatus is subsequently and separately processable as well.

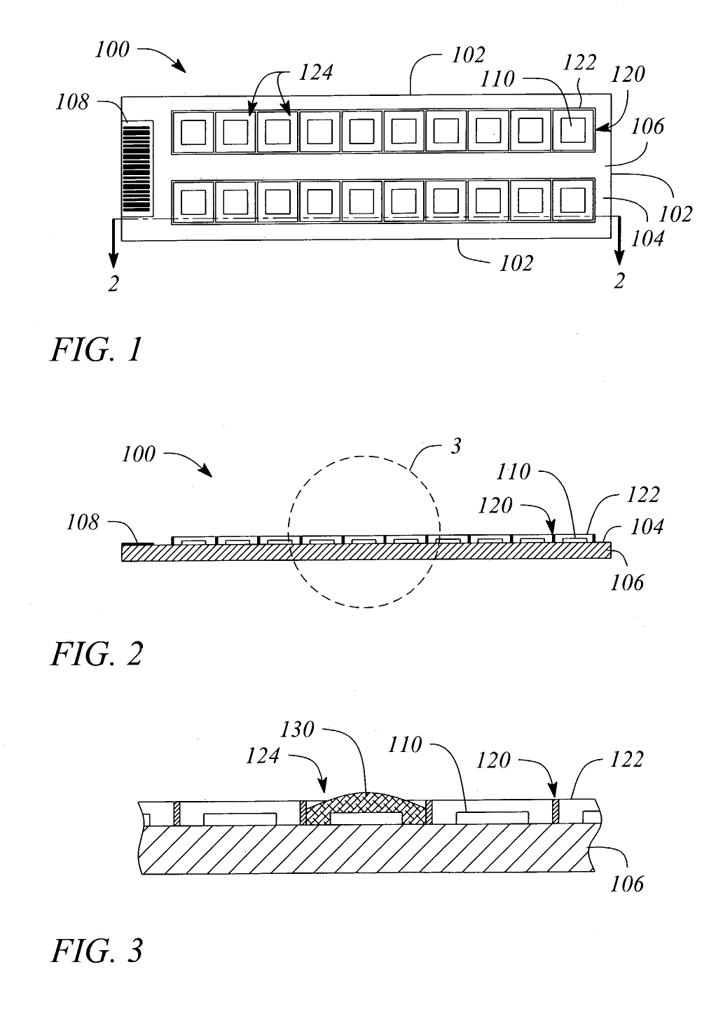

FIG. 1 illustrates a surface or top view of a chemical array apparatus 100 according to an embodiment of the present invention. The apparatus 100 comprises a microarray 110 attached to a surface 104 of a planar array substrate 106. The microarray 110 may be from a plurality of microarrays 110 in a spatially arranged array pattern on the substrate surface. Each microarray 110 is provided in a different spatially addressable region of the planar substrate 106. A microarray 110 comprises a plurality of a chemical or biochemical discrete probe, such as discrete polymer or biopolymer probes, that are spatially arranged in a subarray pattern of chemical features attached to the surface 104 of the planar array substrate 106. Each chemical feature is spatially addressable within the microarray 110. The probes of a given feature may have the same chemical make-up (i.e., monomer sequence) as or a different monomer sequence from the probes of another feature of the same microarray 110. Likewise, the chemical make-up (i.e., monomer sequence of the probes) of a given microarray 110 may be the same as or different from the chemical make-up of another microarray 110 of the plurality.

For example, the plurality of microarrays 110 of the chemical array apparatus 100 of the present invention can be arranged in a 2×10 array pattern on a 2.5 centimeter (cm)×7.5 cm glass support 106 with a modified or derivatized surface 104. Each microarray can comprises one hundred oligonucleotides, for example, representing a limited number of genes (tiled from the 3′ end of each transcript). The oligonucleotides are generated on the modified glass support in 10×10 subarray pattern. At one end of the glass support 106, an identifier or mark 108 is provided that identifies and facilitates distinguishing the glass support 106 from other glass supports. The identifier 108 further facilitates orienting the glass support for processing of the apparatus 100.

FIG. 1 further illustrates that the apparatus 100 further comprises a barrier or seal 120 on the surface 104 of the planar substrate 106 to define physical boundaries around individual microarrays 110. The seal or barrier 120 is also referred to herein as a ‘breakaway seal or barrier’ for reasons described further below. The seal or barrier 120 is provided in a grid pattern that corresponds to and is aligned with the array pattern of microarrays 110. In particular, the grid pattern comprises a plurality of zones 124 in a corresponding array pattern such that a zone 124 of the seal or barrier 120 surrounds a respective microarray 110 on sides of the microarray 110 that are parallel to edges 102 of the substrate 106. However, the seal or barrier 120 does not cover the microarray 110. By ‘does not cover’, it is meant that the seal or barrier 120 does not extend over the microarrays 110 in a plane that is parallel to a plane of the substrate surface 104. The seal or barrier 120 provides each microarray 110 fluid isolation from a remainder of the plurality of microarrays 110.

In some embodiments, the seal or barrier 120 is a physical barrier that is made of a viscous material that has controllable flow characteristics before curing, including but not limited to an elastomer, such as a silicone, an epoxy or any other material with controllable flow characteristics before curing. For examples of materials and their deposition that are applicable to the present invention, please see pending U.S. patent application of Arthur Schleifer entitled “Form in Place Gaskets for Assays” Ser. No. 10/172,850, filed Jun. 14, 2002, or Brown et al., U.S. Pat. No. 5,807,522, both of which are incorporated by reference herein in their entirety. In particular, the seal or barrier material is compatible with, or inert with respect to, the chemical make-up of the microarrays, especially after the barrier material is cured. For example, the material does not interfere with the synthesis, deposition, immobilization, assay or hybridization, and detection or scanning processes, materials, samples and reagents associated with the fabrication and use of the microarrays 110 on the chemical array apparatus 100.

The physical seal or barrier 120 extends approximately perpendicular from the surface 104 of the substrate 106 to create barrier sidewalls 122. Moreover, the seal or barrier 120 is readily manipulable or workable after it is cured. By ‘manipulable’ or ‘workable’ according to the present invention, it is meant that a localized or confined application of pressure to a portion of the cured material affects the breakaway seal 120 at the portion. The portion of the breakaway seal 120 is either the sidewall 122 or a discrete area of the sidewall 122. In particular, the localized pressure causes the seal or barrier 120 to break away from itself, and preferably from the surface 104 of the substrate 106 at the portion. Preferably, the seal or barrier 120 breaks away essentially in the discrete area of the sidewall 122, such that the break includes a nominal amount of an immediately adjacent part of the seal or barrier 120. The broken away portion creates a gap or nick in a sidewall 122 of the seal or barrier 120 in the discrete area. Alternatively, the seal or barrier 120 can break away an amount ranging from the nominal amount up to an amount equivalent to a length or extent of the entire respective sidewall and still be within the scope of the present invention. The reason for the gap will be explained below.

In other embodiments, the seal or barrier 120 is a chemical barrier, such as one that is hydrophobic relative to the synthesis, deposition, immobilization and assay materials, samples and reagents associated with the fabrication and use of the microarrays 110. For examples of materials and methods of forming a chemical barrier that is applicable to the present invention, see Brennan, U.S. Pat. No. 6,210,894 B1, and Alfenito, U.S. Pat. No. 6,355,419 B1, both of which are incorporated by reference herein in their entirety. As described above for the physical barrier 120, the chemical barrier 120 according to the invention is manipulable or workable such that a gap in the chemical barrier is readily creatable in a discrete area of the chemical barrier by either hydrolysis of a linking group that attaches the hydrophobic chemical barrier moiety to the support or the direct chemical modification of the hydrophobic moiety. By way of example, brief exposure of a succinyl linker to a moderate strength acid or base results in the hydrolysis of an ester linkage thereof. A base is preferred as the conditions for hydrolysis are relatively milder.

FIG. 2 illustrates a cross sectional view of the apparatus 100 of FIG. 1 according to an embodiment using the physical seal or barrier 120. The cross section is taken along lines 2-2 in FIG. 1, which is just inside the seal or barrier 120 parallel to one outer or perimeter edge 102 of the planar substrate 106. As mentioned above, the seal or barrier 120 surrounds each microarray 110 but does not cover the microarrays 110. For the embodiment illustrated in FIGS. 1 and 2, the seal or barrier 120 surrounds the microarray 110 on four sides, with one of the sides 122 being cut away in the cross section of FIG. 2. FIG. 2 exemplifies relative dimensional relationships between the physical seal or barrier 120 and the microarray 110. For example, a height of the sidewalls 122 of the physical seal or barrier 120 is greater than a height that the chemical probes of the microarray 110 extend from the surface 104. It should be noted that the apparatus 100 and the elements of the apparatus 100, such as the microarrays 110 and the seal or barrier 120, are not drawn to scale in the Figures provided herein. Moreover, the array pattern of microarrays 110 illustrated can be any variety of array patterns and still be within the scope of the present invention. Likewise, the grid pattern of the seal or barrier 120 can be any corresponding variety of grid patterns and be within the scope of the present invention. The embodiments illustrated in the Figures are exemplary only and not intended to limit the scope of the present invention.

The apparatus 100 is particularly useful for processing, such as assaying, a single or individual microarray 110 of the plurality of microarrays separately from the remaining microarrays 110 of the plurality without contaminating or otherwise affecting the remaining microarrays 110. Moreover, subsequent to assaying the single microarray 110, any one or more of the remaining microarrays 110 can be assayed separately in time without contamination or affect to the remainder. In other words, the apparatus 100 is particularly useful for performing hybridization assays on as few as one microarray 110 of the plurality at a time while not contaminating the other microarrays, thereby rendering the other microarrays available for subsequent assays. Accordingly, each of the microarrays 110 of the plurality is available for processing separately in time, or one at a time. However, it is within the scope of the present invention for more than one microarray 110 to be assayed together. The number of microarrays 110 of the apparatus 100 that are assayed at one time is dependent on the user of the apparatus 100 and is not a limitation herein. The present apparatus 100 advantageously accommodates the processing of as few as one microarray 110 of the chemical array at a time. Moreover, the chemical array apparatus 100 of the present invention advantageous provides a user of the apparatus 100 diverse and/or multiple types of uses for the microarrays 110 of the apparatus 100.

During a hybridization assay of a particular individual microarray 110 (hereinafter a ‘first’ microarray 110 for simplicity), according to the present invention, a fluid test sample 130 is deposited directly on the first microarray 110 within a first zone 124 created by the seal or barrier 120 surrounding the first microarray 110. FIG. 3 is a magnified view illustrating the apparatus 100 from FIG. 2 during an assay. The magnified portion is an encircled area labeled ‘3’ in FIG. 2. The fluid test sample 130 is deposited using a pipette or other means for depositing a fluid sample, which are known in the art, that provide for relatively precise deposition of a predefined amount of a fluid test sample or reagent to a particular location. The seal or barrier 120 retains the fluid test sample 130 within the first zone 124. The predefined amount or volume of the test sample 130 ensures that the test sample 130 will not overflow into an adjacent zone that includes one of the remaining microarrays 110 of the plurality.

Depending on the embodiment, the apparatus 100 may further comprise a cover that is applied over the zone 124 having the fluid sample 130 therein in order to reduce the rate of evaporation of the test sample solution 130 during processing. Moreover, the apparatus 100 may further comprise a removable cover that is applied over the remaining microarrays 110 of the plurality to assist with avoiding fluid contamination to the remaining microarrays 110, as well as other contamination thereto from handling and storage of the apparatus 100. A discussion of using a cover is further described below with respect to a system 200 for processing a microarray of a chemical array in accordance with another aspect of the present invention.

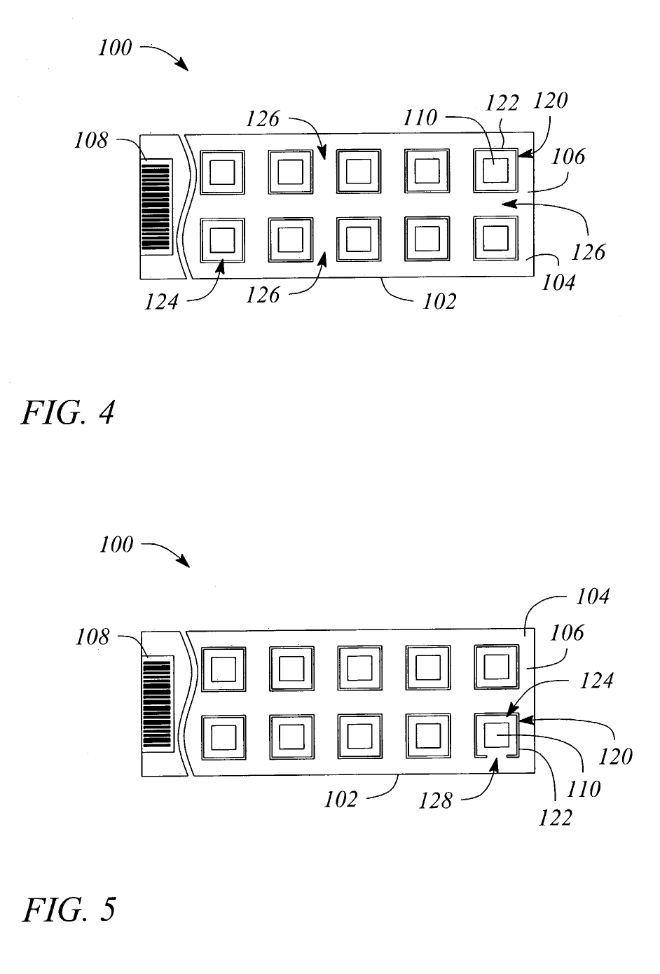

In other embodiments of the physical seal or barrier 120, the grid pattern preferably includes a channel 126. The channel 126 provides a space for fluid samples or reagents to collect or flow during processing, which essentially prevents contamination of unprocessed microarrays 110. In some situations, the channel 126 also provides an overflow path in the undesirable event that a predefined volume of the fluid deposited over the microarray 110 exceeds the volume space of the zone 124. FIG. 4 illustrates a top or surface view of the apparatus 100 according to some embodiments wherein the grid pattern of the seal or barrier 120 includes a channel 126. The channel 126 is created by forming the grid pattern comprising sidewalls 122 that are not shared between adjacent zones 124, but instead are physically separate. The space between adjacent sidewalls 122 that separate adjacent microarrays 10 forms the channel 126. In still other embodiments not shown, the grid pattern comprises a combination of both shared and separate sidewalls, such that an embodiment of the channel 126 is created.

The seal or barrier 120 retains the fluid sample 130 during a hybridization time, for example, which can be any length of time known in the art for a hybridization assay incubation time. When the hybridization time is up, at least a portion of a seal or barrier 120 sidewall 122 is manipulated according to the invention by breaking away a piece of the sidewall 122 material from the zone 124. The sidewall 122 that is manipulated is a sidewall 122 that is not shared by an adjacent microarray, especially when the adjacent microarray is not being processed at the same time as the isolated microarray 110. Further, the sidewall 122 that is manipulated is preferably closest or adjacent to and facing an outer or perimeter edge 102 of the substrate 106. By ‘closest or adjacent to’ it is meant that when the gap is created in the sidewall 122, a relatively unhindered flow or drainage path to the respective substrate perimeter edge 102 is provided for the fluid sample 130. By ‘relatively unhindered’ it is meant either a direct flow path to the respective edge 102, a short flow path, or a flow path that is less likely to contaminate other microarrays, for example. The broken away piece provides a gap 128 in the sidewall 122. FIG. 5 illustrates a surface or top view of the apparatus 100 having a sidewall 122 broken away so as to form a gap 128 in the seal or barrier 120 according to an embodiment of the present invention.

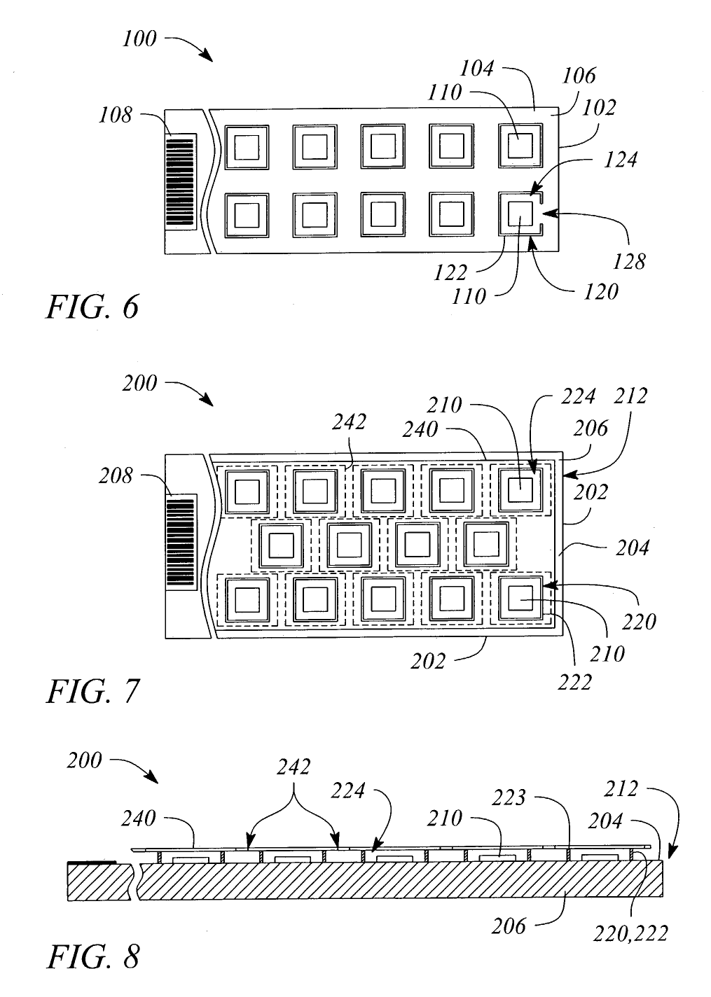

In the embodiment illustrated in FIG. 5, some zones 124 have more than one sidewall 122 that can be manipulated to form the gap 128 that meets the criteria stated above. FIG. 6 illustrates the apparatus 100 of FIG. 5 with a different sidewall 122 being manipulated to form the gap 128. In both FIGS. 5 and 6, the manipulated sidewalls 122 are not shared by adjacent microarrays and both are facing an outer or perimeter edge 102 of the substrate 106, and preferably are closest to the edge 102, relative to the other sidewalls of the respective zone 124. Preferably, the manipulated sidewalls 122 are not facing an adjacent microarray 110. In either embodiment, the formation of the gap 128 provides a path for the release or removal of the fluid test sample 130 that is directed away from the remainder of the microarrays 110 on the chemical array apparatus 100. It should be noted that the sidewall 122 that is manipulated to drain a fluid from the respective microarray 110 is dependent on a number of factors, some of which are user dependent. For example, the number of microarrays being assayed together will influence the user's choice of the sidewall 122 to manipulate during processing. Moreover, if the array apparatus 100 further comprises a cover, as further described below, then the cover may influence the user's choice of the sidewall 122 to manipulate, for example. Therefore, whether an unshared or shared sidewall 122 is manipulated to create the gap 128, and/or whether the sidewall 122 that is manipulated to create the gap 128 faces or is closest or adjacent to a perimeter edge 102 of the planar substrate 106 are not features intended to limit the scope of the present invention. Those skilled in the art may use different or combinations of sidewall 122 configurations to manipulate for a particular application that are all within the scope of the present invention.

Advantageously, the apparatus 100 can be tilted at an angle such that the release of the fluid test sample 130 through the gap 128 is encouraged or assisted. Further washing or rinsing procedures associated with a hybridization assay can be performed while the apparatus 100 is so tilted. A pipette or other means known in the art for applying a directed and confined amount of a fluid rinse or wash reagent to a particular microarray 110 is used to wash the first microarray 110 as a part of the process or assay.

Further advantageously, the first microarray 110 of the apparatus 100 can be scanned using conventional scanning equipment to detect signals from the hybridized target sample as a part of the assay of the first microarray 110. Scanning the first microarray 110 does not interfere with or damage the remaining microarrays 110 on the apparatus 100. Moreover, any of the remaining microarrays 110 can be subsequently assayed one at a time as the first microarray 110 was assayed, or in a larger group, using a fluid test sample that is either the same as or different from the test sample 130 used on the assayed first microarray 110. Still further, a user can use the remaining microarrays 110 for any other purpose desired by the user subsequent to the use of the first microarray 110.

FIG. 7 illustrates a top or surface view of an embodiment of a system 200 for processing a microarray 210 of a chemical array 212 according to another aspect of the present invention. The system 200 comprises a chemical array 212 that comprises a microarray 210 attached to a surface 204 of a planar substrate 206. The microarray 210 typically is from a plurality of microarrays 210 attached to the substrate surface 204 in a spatially addressable array pattern. The chemical array 212 further comprises a seal or barrier 220 on the surface 204 of the array substrate 206 in a grid pattern to separately surround the microarrays 210. The chemical array 212 of the system 200 is essentially the same as or at least similar to the chemical array apparatus 100 described above. The system 200 further comprises a cover 240 extending over the plurality of microarrays 210. The cover 240 is in contact with an outermost or uppermost edge surface 223 of the seal or barrier 220, as further illustrated in FIG. 8. FIG. 8 illustrates a cross sectional view of the system 200 according to an embodiment. As illustrated in FIG. 8, sidewalls 222 of the seal or barrier 220 have a height that is higher than the chemical probes of the microarray 210. The height of the seal or barrier 220 facilitates preventing the cover 240 from contacting the underlying microarray probes. The cover provides protection to the plurality of microarrays 210 when handling the chemical array 212.

The cover 240 is made from a material that forms a thin sheet or film, such as a cellophane or other thin plastic film, or a foil. The cover 240 may be transparent or opaque, or have any clarity in between. The cover 240 is tacked down onto or otherwise adheres to the seal or barrier edge surface 223 using one or more of an adhesive, a tacky seal or barrier material and/or cover material, and an electrostatic attraction between the cover 240 and the seal or barrier 220, for example, depending on the embodiment. When an adhesive is used, the adhesive is provided to a surface of the cover 240 that faces and contacts the seal or barrier 220 edge surface 223. The adhesive may be provided to the surface of the cover 240 at discrete positions aligned with the seal or barrier 220, or a continuous strip of adhesive may be provided that is aligned with the seal or barrier 220. Likewise, an adhesive may be provided on the seal or barrier 220 at discrete positions or as an adhesive strip to adhere to the cover 240 during the assembly of the system 200.

The cover 240 is a malleable or stretchable film that spreads out to form a taut or stiff cover when applied to the chemical array 212, such that the cover 240 does not sag or droop into zone 224 cavities created by the seal Or barrier 220. Alternatively, the cover 240 may be a rigid or semi-rigid plastic sheet or plate, according to some embodiments. The cover 240 comprises means for readily separating 242 a section of the cover 240 from other sections remaining on the seal or barrier 220 without damaging the remaining sections. The means for readily separating 242 comprises a scored pattern 242, such as a pattern of perforations 242 through the cover 240, which renders the separation of a cover section from other cover sections easily achievable without damage. The scored pattern 242 corresponds to or is relatively aligned with the grid pattern of the seal or barrier 220. The scored pattern 242 provides for removal with relative ease of a section 244 of the cover 240 from a zone 224 of the seal or barrier 220 surrounding a particular microarray 210 while leaving a remainder of the cover 240 over the remaining microarrays 210 intact and without damaging the underlying remaining microarrays 210. The removed section 244 of the cover 240 is readily removable from a remainder of the cover 240 along a corresponding portion of the scored pattern 242 to expose an individual microarray 210. Each section 244 of the cover 240 is readily removable by peeling the cover section 244 away from the seal or barrier zone 224 and separating the section 244 along the corresponding portion of the scored pattern 242, such as by tearing at respective perforations. These separated sections 244 of the cover 240 can be retained for later use. For example, a microarray 210 is uncovered by separating a respective section 244 of the cover 240 from a respective zone 224. The uncovered microarray 210 is processed, such as with a fluid reagent or fluid target sample solution during an assay. After the fluid solution is applied, the respective separated section 224 of the cover 240 can be placed on top of a fluid reagent or fluid sample solution (i.e., floated on the solution) to form a partial seal or cover. The created partial seal or cover helps to reduce the rate of evaporation of the solution, among other things, during exposure to the atmosphere.

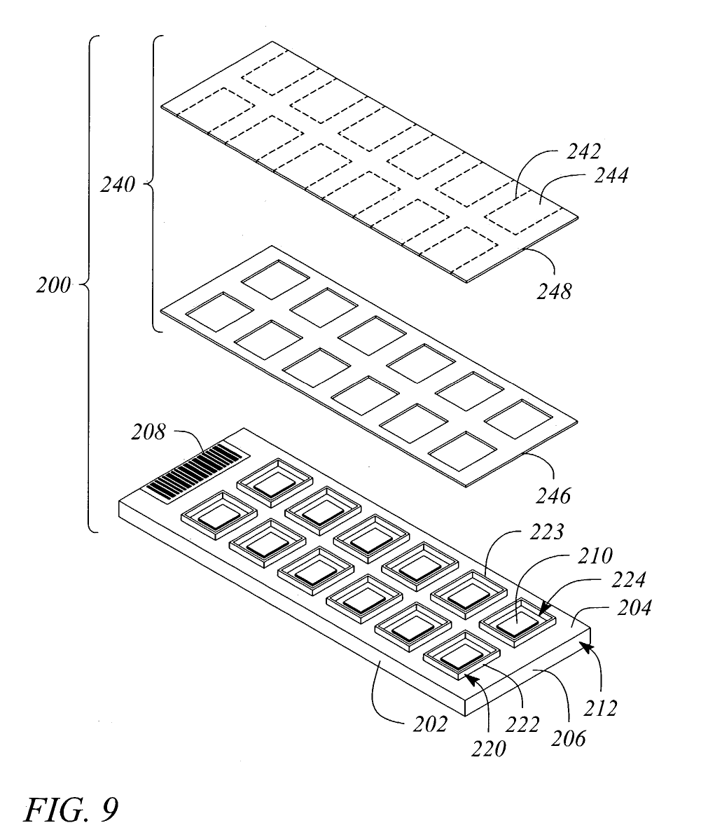

In some embodiments, the cover 240 is a two-part cover, such as that illustrated in a perspective view of the system 200 in FIG. 9 according to an embodiment. The two-part cover 240 comprises a rigid or semi-rigid grid frame 246 made from a thin plastic sheet, for example. The grid frame 246 comprises a plurality of grid frame units in a grid frame pattern that corresponds to the grid pattern of the seal or barrier 220. Each grid frame unit overlies a different seal or barrier zone 224. The grid frame 246 securely adheres to the seal or barrier uppermost edge surface 223. The two-part cover 240 further comprises a thin flexible film 248 overlying the grid frame 246. The flexible film 248 is solid but for a scored pattern 242 therein. The flexible film 248 loosely adheres to the grid frame 246 for removal relative to the adhesion between the grid frame 246 and the seal or barrier 220. The flexible film 248 essentially peels away from the grid frame 246 in sections 244 along the scored pattern 242 aligned over the grid frame units and the corresponding zones 224 created by the seal or barrier 220. A section 244 of the flexible film 248 is readily removable from the system 200 one at a time to uncover a single underlying microarray 210 for processing according to the present invention.

Advantageously, the system 200 provides more protection to the plurality of microarrays 210 than the chemical array apparatus 100 alone. Physical damage is less likely to occur to the covered microarrays 210 during handling and storage. Further, contamination to the covered microarrays 210 from processing a single microarray 210 of the plurality is less likely to occur.

When a section 244 of the cover is removed, the system 200 provides for fluid access to the uncovered microarray 210. As described above for the seal or barrier 120 of the chemical array apparatus 100, the seal or barrier 220 retains a fluid sample or reagent that is used to process an uncovered microarray 210. Further, the seal or barrier 220 is manipulable, such that when localized or confined pressure is deliberately applied to a discrete location on the seal or barrier 220, the seal or barrier 220 breaks away at the location to create a gap in the seal or barrier 220 at the discrete location. When the seal or barrier 220 breaks away, the seal or barrier material breaks away from itself and further, may break away from the surface 204 of the substrate 206.

When a gap is deliberately formed in the seal or barrier 220; the fluid reagent or sample is released and the uncovered microarray 210 may be further processed, such as by washing or rinsing the microarray 210, drying the uncovered and processed microarray 210, as appropriate, and scanning the uncovered and processed microarray 210 using conventional washing, drying and scanning techniques known in the art.

The remaining cover 240 protects the remaining microarrays 210 from contamination during processing of the uncovered microarray 210 with fluid samples and reagents. The remaining cover 240 may further provide protection from the scanning process, such as blocking a scanning light from penetrating the cover 240, depending on the embodiment.

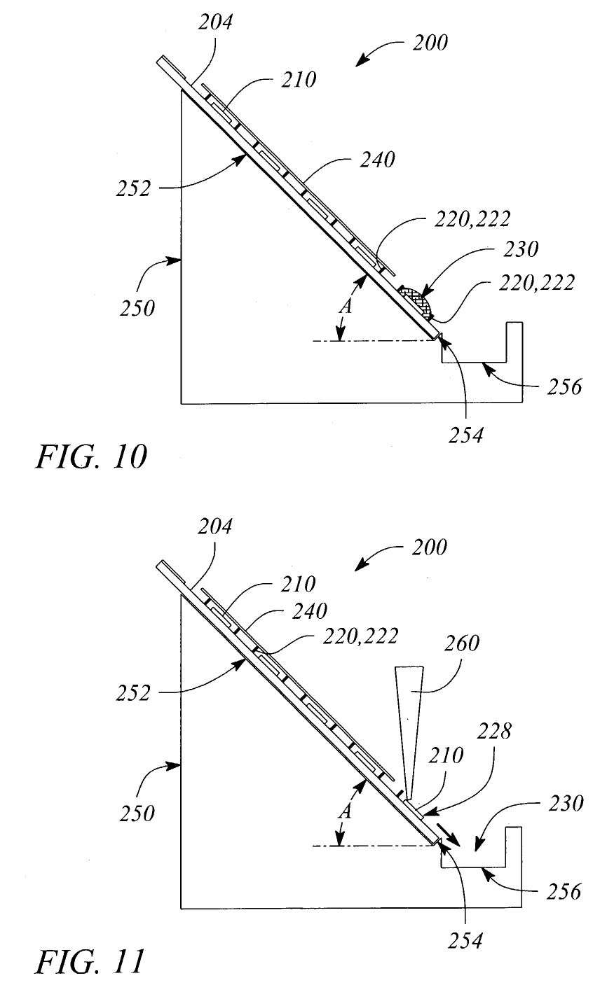

In some embodiments, the system 200 further comprises a fixture that receives and holds the chemical array 212 or apparatus 100 at a fixed angle or incline for processing. FIG. 10 illustrates a fixture 250 that comprises an inclined plane 252 having a lip or shelf 254 at a lower end of the incline plane 252. The chemical array 212 including the remaining cover 240 is placed against the inclined plane 252 of the fixture 250 with the substrate surface 204 facing away from the inclined plane 252. The chemical array 212 is positioned in the fixture 250, such that a sidewall 222 of the seal or barrier 220 surrounding the uncovered microarray 210 faces the lower end adjacent to the shelf 254. As mentioned above for the chemical array apparatus 100, the sidewall 222 is facing an outer or perimeter edge 202 of the substrate 206 and preferably, is close to or adjacent to the edge 202 and/or faces away from the remaining microarrays 210. The fixture 250 optionally comprises a fluid basin 256 below or adjacent to the shelf 254. The fluid basin 256 receives waste fluids, such as the fluid test sample 230 and other reagents or wash solutions used during processing the uncovered microarray 210. The chemical array 212 is placed in the fixture 250 either before or after the breakaway seal or barrier 220 is broken. Surface tension between the fluid reagent or sample 230 and the seal or barrier 220 prevents the fluid sample 230 from overflowing the sidewall 222 of the seal or barrier 220 while in the tilted position before the gap is created.

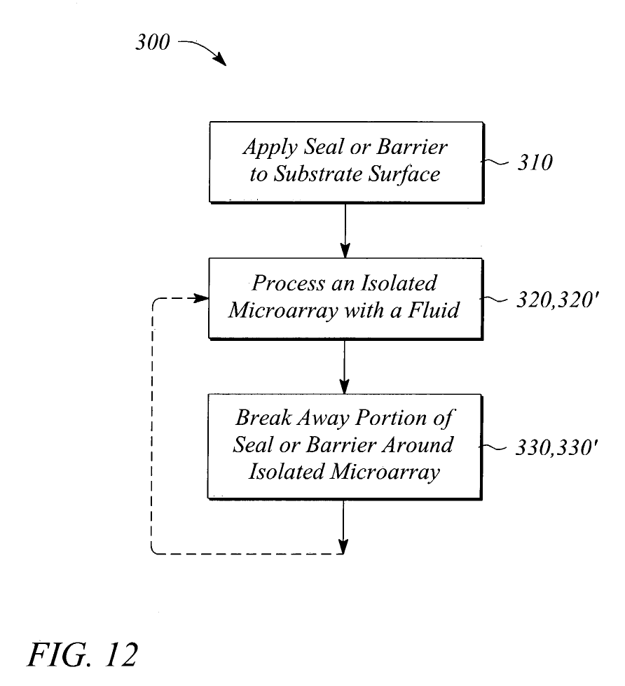

In some of these embodiments, the system 200 further comprises a tool (not illustrated) for deliberately applying localized pressure to the sidewall 222 of the breakaway seal or barrier 220 to create the gap. The tool has a rigid tip that is a pointed or a tapered blunt tip, and can be a conventional pipette. The tip of the tool is used to apply the localized pressure that breaks the seal or barrier 220. Alternatively, the tool can be a device having a sharper tip, such as a laboratory knife, that has a rigid blade with a tapered tip. In some of these embodiments, the system 200 may further comprise a dispenser for applying a controlled or directed stream of a wash solution or other fluid reagent to the uncovered microarray 210. FIG. 11 illustrates the system 200 including a dispenser 260 according to some embodiments of the present invention. Only a tip portion of the dispenser 260 is illustrated in FIG. 11 along with a single-headed straight arrow showing the direction of fluid flow into the basin 256. In some embodiments, the dispenser 260 is a conventional laboratory micropipette. In an alternative embodiment, the dispenser 260 is part of dispensing equipment (not shown) that provides for automatic and/or semi-automatic control of the dispenser 260. The control provided by this alternative embodiment includes control of one or more of the type of fluid dispensed, the quantity of fluid dispensed, and the positioning and/or movement of the dispenser 260 over the processed microarray 210.

FIG. 12 illustrates an embodiment of a method 300 of assaying a microarray of a chemical array of microarrays according to another aspect of the present invention. The method 300 comprises applying 310 a seal or barrier material to a surface of a planar array substrate. The seal or barrier material is applied 310 in a grid pattern that comprises a plurality of zones arranged in an array pattern. A zone of the barrier grid array pattern has sufficient dimension to ultimately surround and isolate a microarray of an array pattern of microarrays from one another within the barrier grid pattern. The method 300 further comprises processing 320 a respective isolated microarray of the array pattern within a respective zone of the grid pattern with a fluid. Processing 320 includes depositing a fluid reagent or sample on the respective microarray (also referred to herein as a first isolated microarray) of the array pattern. The respective first zone of the seal or barrier grid pattern surrounding the first isolated microarray retains the fluid. The method 300 further comprises breaking away 330 a portion of the seal or barrier of the respective zone to create a gap, such that the fluid is released from the respective zone through the gap.

In some embodiments, the method 300 may further comprise one or more of rinsing the respective first microarray with a wash solution that drains through the gap, drying the first microarray, and scanning the first microarray using scanning equipment to evaluate the processing 320.

In some embodiments, the isolated microarray is processed 320 by performing a hybridization assay with a fluid test sample. In this embodiment, processing or assaying 320 comprises dispensing a first fluid test sample on the first isolated microarray of the array pattern, and incubating the first fluid test sample with the first isolated microarray for a period of time. The first fluid test sample is intended to hybridize to biopolymer material of the first isolated microarray, for example. The first zone retains the first fluid test sample during incubation and until the gap is created. In the hybridization assay embodiments, the hybridized microarray is rinsed or washed, optionally dried, and further scanned to detect and determine the results of the hybridization assay.

The method 300 according to some embodiments further comprises storing the chemical array of microarrays in a dry and stable environment, such as a nitrogen or inert gas chamber, until processing of another isolated microarray of the chemical array is desired. The other isolated microarray is a microarray that is surrounded by an intact zone of the seal or barrier. Alternatively, the chemical array is not stored for a substantial amount of time. In the alternative embodiment, and further in the stored array embodiments after the storage period is over, the method 300 further comprises processing 320′ a second isolated microarray. In effect, the second isolated microarray is processed 320′ after the first isolated microarray was processed 320 and further, after the seal or barrier around the first isolated microarray is broken away 330 to release the first fluid.

The second isolated microarray is processed 320′ in a hybridization assay or another type of assay or another process, which is dependent on the user of the chemical array of microarrays. The processing 320′ of the second isolated microarray comprises depositing a second fluid, such as a fluid reagent or a fluid test sample, to the second isolated microarray. A second zone of the barrier grid pattern surrounds the second isolated microarray and retains the deposited second fluid. Subsequently, a portion of seal or barrier of the second zone is broken away 330′ to release the second fluid. If the processing 320′ is a hybridization assay, the processing 320′ includes respective incubation, rinsing, optional drying, and further scanning, as described above for the assaying embodiments of the first isolated microarray.

The application 310 of the seal or barrier to the substrate surface comprises applying the grid pattern such that zones are created that comprise sidewalls that surround each microarray on sides of the microarray, as opposed to covering the microarray. The sidewalls of a zone, and preferably each zone, are approximately parallel to outer or perimeter edges of the planar array substrate. Depending on the embodiment, a sidewall of the seal or barrier between two adjacent microarrays is one or more of shared and unshared (i.e., separate), such that a combination of shared sidewalls and unshared sidewalls between adjacent zones is within the scope of the invention.

According to the present invention, the array pattern of microarrays is provided on the array substrate before the seal or barrier is applied 310 in some embodiments. However in other embodiments, the method 300 further comprises providing, such as by attaching, a plurality of microarrays to the surface of the array substrate in the array pattern. The plurality of microarrays are provided either before or after the application 310 of the seal or barrier grid pattern, according to these other embodiments. The array pattern of microarrays is a spatially addressable array pattern that corresponds to the grid pattern. By ‘corresponds’, it is meant that a microarray, and preferably each microarray, of the plurality is within (or surrounded by) a different zone of the grid pattern. A microarray comprises a plurality of a chemical or biochemical material, such as polymers or biopolymer probes, spatially arranged as addressable features in a subarray pattern on the substrate surface. For microarrays of oligonucleotide probes, the microarrays are added to the substrate either by in situ synthesis of the probes or as presynthesized probes that are attached or immobilized on the substrate surface. Any of the methods known in the art for in situ synthesis and/or attachment of oligonucleotide probes to an array may be used for the present invention.

The seal or barrier is applied 310 to the substrate surface using a handheld syringe of the seal or barrier material, or another manual means for dispensing. Alternatively, the seal or barrier is applied 310 using automated or semi-automated dispensing equipment that controls at least one or both of the amount of material dispensed and the position of the grid pattern of the dispensed material relative to the substrate. Those skilled in the art are familiar with conventional dispensing means that are within the scope of the present invention. Moreover, both A. Schleifer, Ser. No. 10/172,850, and Brown et al., U.S. Pat. No. 5,807,522, both cited supra, disclose dispensing means and methods that would be useful for the present invention.

When the portion of the seal or barrier is broken away 330 from the respective zone to create the gap, the respective zone contains the isolated microarray and preferably the fluid deposited during processing 320, 320′. However depending on the processing 320, 320′ performed by the user of the chemical array, it may be desirable to break away 330 the portion of the seal or barrier prior to adding the fluid to the respective zone. In this way, the fluid would be able to drain from the zone while it is being deposited. Whether the seal or barrier portion is broken away 330 prior to or after a fluid is added to the respective zone during processing 320, 320′ is user dependent and both are within the scope of the present invention.

Breaking away 330 a portion of the seal or barrier of the respective zone comprises holding the array substrate at an incline angle. The incline angle is that which will allow the fluid to readily drain through the gap when created, but not allow the fluid to drain or overflow the seal or barrier absent the gap. In other words, the incline angle is not so steep such that gravity overcomes the surface tension holding the fluid in the respective zone. If the fluid flows over the respective zone sidewalls then the incline angle is too steep. The incline angle ‘A’ will range from approximately 15° to approximately 60°, for example, from a horizontal line illustrated in FIGS. 10 and 11 as a dashed horizontal line, depending on the substrate surface. However, the incline angle range is not a limitation to the present invention and will depend on the particular fluids applied by the user during processing. Any angle that achieves the goals above is within the scope of the present invention. For example, a generally hydrophilic surface may dictate the use of an angle that assures that the solution is shed relatively quickly from the surface, such that the solution does not puddle or dry in place, for example. Depending on the surface hydrophilicity, the incline angle typically ranges from about 30° to about 60°. However, a generally hydrophobic surface may dictate the use of an angle that assures that the solution spreads as it travels over the surface, such that the solution does not run as a stream in a narrow path, for example. Depending on the surface hydrophobicity, the incline angle ranges from about 15° to about 45°.

Breaking away 330 further comprises orienting the array substrate such that an outer or perimeter substrate edge adjacent to the respective zone is at lowest position when inclined. The position of the adjacent perimeter substrate edge is lower relative to the other perimeter edges of the planar substrate. Further, a sidewall of the respective zone faces the adjacent perimeter substrate edge that is in the lowest position. Moreover, breaking away 330 further comprises applying localized or confined pressure to a sidewall of the respective zone that preferably faces and is adjacent to the lowest perimeter substrate edge of the planar substrate. The localized pressure is applied to a portion of the sidewall to create the gap in the sidewall. When localized pressure is applied, a piece of the sidewall breaks away from the remaining sidewall. Preferably although not required, the sidewall piece also breaks away from the surface of the planar substrate. Essentially, the planar array substrate is tilted and oriented in a direction such that the fluid retained by the respective zone will drain through the created gap preferably directly off the array substrate via the closest or adjacent or facing lowest perimeter edge. The pressure is preferably applied to the sidewall closest or adjacent to and facing the lowest perimeter substrate edge to reduce a drainage path that the fluid will take from the created gap off the planar array substrate. The created gap allows the fluid to drain from the zone along a drainage path off the array substrate. More preferably, the gap is created in the zone sidewall facing away from adjacent microarrays. Moreover, the fluid will drain through the gap along a drainage path that preferably is directed away from other microarrays of the array pattern to avoid contamination of the other microarrays.

In some embodiments, holding the array substrate at an incline angle comprises inserting the array substrate in a fixture that comprising an inclined surface having a shelf. The shelf prevents the array substrate from sliding in the direction of the incline. The lowest perimeter edge of the substrate is adjacent to the shelf. The fixture may further comprise a fluid collection basin adjacent to or below the shelf. The fluid collection basin has an opening for receiving the fluid. The gap can be created while the array substrate is placed in the fixture to stabilize the array substrate while point pressure is applied to the respective zone sidewall. The gap created in the respective zone faces the collection basin opening.

In some embodiments of the method 300, the method further comprises applying a cover over (i.e., covering) the planar array substrate. The cover is a sheet or film of material that is described further above with respect to the cover 240 of the system 200. The cover is applied after the seal or barrier is applied 310 to the substrate. Preferably, the cover is applied after the plurality of microarrays is provided to the substrate surface. When the cover is applied, it is adhered to and in contact with the seal or barrier. The zone sidewalls of the seal or barrier have a height measured from the substrate surface that is higher than a height that the microarrays extend off the substrate surface. The sidewalls further have upper edges (i.e., a top of the sidewalls) that are opposite to the substrate surface (i.e., opposite to a bottom of the sidewalls that are adjacent to the substrate surface). The cover is applied to the upper edges of the sidewalls. The sidewalls have sufficient height and the cover is sufficiently taut, such that the cover does not contact the microarrays when applied to the array substrate. In these embodiments, processing 320, 320′ an isolated microarray further comprises removing the cover to access the isolated microarray before depositing the fluid. Preferably, the cover is selectively removed, such that only a section of the cover that corresponds to (i.e., covers) the respective zone and the isolated microarray to be processed 320, 320′ is removed.

Advantageously, the applied cover provides protection from damage and contamination to the microarrays during handling and storage. The seal or barrier further provides protection from fluid contamination to a remainder of the microarrays during processing 320, 320′ of as few as a single microarray. Further, the applied cover provides added protection to the remaining microarrays while individual microarrays are processed. Moreover, the section of the cover that is removed from the respective zone to process the isolated microarray advantageously can be floated on the surface of the fluid sample or solution applied in the respective zone to reduce evaporation of the solution during processing 320, 320′.

Thus there have been described several embodiments of a novel chemical array apparatus, a system for and a method of processing a microarray of a chemical array of microarrays. It should be understood that the above-described embodiments are merely illustrative of some of the many specific embodiments that represent the principles of the present invention. Clearly, those skilled in the art can readily devise numerous other arrangements without departing from the scope of the present invention.

What is claimed is:

1. A chemical array apparatus comprising:
a breakaway seal applied to a surface of a planar substrate, the breakaway seal surrounding a microarray attached to the substrate surface, wherein a portion of the breakaway seal is removable to create a gap in the breakaway seal.

2. The chemical array apparatus of claim 1, wherein the breakaway seal comprises one or both of a physical seal and a chemical seal.

3. The chemical array apparatus of claim 1, wherein the breakaway seal provides fluid isolation to the microarray.

4. The chemical array apparatus of claim 3, wherein the breakaway seal retains a fluid with the microarray until the gap is created, the gap being created on the portion of the breakaway seal that provides a drainage path for the fluid to a perimeter edge of the planar substrate.

5. The chemical array apparatus of claim 3, wherein the fluidly isolated microarray is from a plurality of microarrays attached to the surface of the substrate, and wherein during an assay of the isolated microarray, the breakaway seal retains a fluid with the isolated microarray, such that a remainder of the plurality of microarrays is unaffected by the assay of the isolated microarray.

6. The chemical array apparatus of claim 1, wherein the microarray is from a plurality of microarrays attached to the surface of the substrate, and wherein the breakaway seal provides the plurality of microarrays fluid isolation from one another.

7. The chemical array apparatus of claim 1, wherein the breakaway seal comprises a plurality of sides, the microarray being surrounded by respective sides of the breakaway seal to provide fluid isolation, the respective sides that surround a microarray comprise one or both of a side shared by an adjacent microarray and a side unshared by an adjacent microarray.

8. The chemical array apparatus of claim 7, wherein the gap is created on a respective side surrounding the microarray that is unshared by an adjacent microarray.

9. The chemical array apparatus of claim 1, wherein the breakaway seal forms a channel or path located between adjacent microarrays on the substrate surface.

10. The chemical array apparatus of claim 1, wherein the microarray is from a plurality of microarrays, the plurality of microarrays being arranged in an array pattern that is spatially addressable, the microarray of the plurality comprising a plurality of a chemical or biochemical polymer attached to the substrate surface in a spatially addressable subarray pattern, the breakaway seal having a grid pattern that corresponds to the array pattern, the grid pattern comprising a plurality of zones, the microarray being surrounded by a respective zone of the breakaway seal.

11. The chemical array apparatus of claim 1, wherein the breakaway seal has physical sidewalls that extend a height from the substrate surface, the height being greater than a height that the microarray extends from the substrate surface, the sidewalls being capable of retaining a fluid with the microarray.

12. The chemical array apparatus of claim 1, wherein the breakaway seal is formed by changing a chemical characteristic of the substrate surface along sides surrounding the microarray, the changed characteristic of the substrate retaining a fluid with the microarray using one or both of a hydrophobic effect and a hydrophilic effect.

13. The chemical array apparatus of claim 1, further comprising:
a removable cover extending over and in contact with the breakaway seal to enclose or shield the microarray.

14. The chemical array apparatus of claim 13, wherein the microarray is from a plurality of microarrays attached to the substrate surface in an array pattern, the cover shielding the plurality of microarrays, the cover optionally being selectively removable in sections to uncover a respective microarray relative to a remainder of the plurality of microarrays, the cover being intact over the remainder of the microarrays when a section of the cover is selectively removed.

15. A system for processing a microarray of a chemical array comprising:
a chemical array that comprises a microarray attached to a surface of a planar substrate;
a breakaway seal provided on the planar substrate to surround the microarray, wherein a portion of the breakaway seal is removable to create a gap in the breakaway seal; and
a removable cover extending over and in contact with the breakaway seal to shield the microarray.

16. The system of claim 15, wherein the microarray is from a plurality of microarrays attached to the substrate surface in a spatially addressable array pattern, the breakaway seal comprising sidewalls in a grid pattern that form zones, the grid pattern corresponding to the array pattern.

17. The system of claim 15, wherein the breakaway seal is dimensioned to reduce contact between the cover and the microarray.

18. The system of claim 15, wherein the removable cover is taut over the breakaway seal to reduce contact between the cover and the microarray.

19. The system of claim 15, wherein the cover is removable by peeling the cover away from the breakaway seal.

20. The system of claim 16, wherein the cover is selective removably from the breakaway seal, such that when a section of the cover is selectively removed to expose a respective microarray, a remainder of the cover that shields a remainder of the plurality of microarrays remains intact and unaffected by the selective removal of the section.

21. The system of claim 20, wherein the selectively removable cover comprises a scored pattern corresponding to the grid pattern of the breakaway seal, such that the section of the cover is selectively removable by separating the section along respective scoring of the scored pattern.

22. The system of claim 21, wherein the section of the cover is further selectively removable by peeling the section away from the breakaway seal.

23. The system of claim 16, wherein the cover comprises a film layer and a grid frame layer, the grid frame layer having a plurality of grid frame units arranged in a frame grid pattern, the frame grid pattern corresponding to the grid pattern of the breakaway seal, the grid frame layer being adjacent and securely attached to the breakaway seal, the film layer overlying the grid frame layer, the film layer being readily separable from the grid frame layer relative to the secure attachment of the grid frame layer to the breakaway seal.

24. The system of claim 23, wherein the film layer comprises scoring in a scored pattern, the scored pattern corresponding to the grid frame pattern, the film layer being selectively separable from the grid frame layer in sections along the scoring, such that when a section of the film layer is removed, the section is peeled from a respective grid frame unit and separated from a remainder of the film layer along a portion of the scoring, the removed section exposing a underlying microarray that is otherwise surrounded by sidewalls of a respective zone of the breakaway seal and the respective grid frame unit, and the remainder of the film layer being unaffected by the removal of the film layer section.

25. The system of claim 16, wherein a zone of the grid pattern surrounds and provides fluid isolation to a respective microarray from other microarrays of the plurality, the removable cover being selectively removable from the zone to provide fluid access to the respective microarray that is otherwise surrounded by respective sidewalls of the breakaway seal, and
wherein an unremoved portion of the cover remains intact and provides the other microarrays protection from physical damage and fluid contamination.

26. The system of claim 25, wherein during an assay of the respective microarray, a respective section of the cover is selectively removed from the zone to expose the respective microarray, a fluid deposited on the exposed respective microarray being retained by the respective sidewalls of the zone.

27. The system of claim 26, wherein during the assay of the isolated microarray, the removed cover section is applied over the deposited fluid retained by the respective sidewalls to help shield the fluid during the assay.

28. The system of claim 27, wherein further during the assay, localized pressure is deliberately applied to a sidewall of the respective sidewalls of the zone to create the gap in the breakaway seal, the deposited fluid being released through the created gap.

29. The system of claim 15, wherein the portion of the breakaway seal is removed by deliberately applying localized pressure to a sidewall of the breakaway seal, such that the portion breaks away to create the gap in the sidewall.

30. The system of claim 15, further comprising a fixture having a planar inclined surface and a shelf, the inclined surface and the shelf supporting the chemical array during an assay of the microarray.

31. The system of claim 16, wherein the removable cover is adhered to the breakaway seal using one or more of an adhesive at between the cover and an edge surface of a sidewall of the breakaway seal, an electrostatic attraction between the breakaway seal and the cover along the edge surface of a sidewall, and an adhesive strip in an adhesive grid pattern similar to the breakaway seal grid pattern between the cover and the edge surface of the sidewalls.

32. The system of claim 16, wherein the microarray comprises a plurality of a chemical or biochemical polymer attached to the substrate surface in a spatially addressable subarray pattern.

33. A method of processing a microarray of a chemical array of microarrays comprising:
applying a breakaway seal to a surface of a planar substrate to ultimately surround a microarray on the planar substrate;
processing the microarray with a fluid that is deposited on the microarray, the breakaway seal retaining the fluid with the microarray; and
breaking away a portion of the breakaway seal that retains the fluid with the microarray, the broken away portion creating a gap in the breakaway seal, the gap providing an exit for the release of the fluid from the microarray.

34. The method of claim 33, further comprising attaching a plurality of microarrays to the substrate surface in an array pattern either before or after the breakaway seal is applied, the microarray being from the plurality, the array pattern being spatially addressable, the microarray comprising a plurality of a chemical or biochemical polymer arranged in a subarray pattern that is spatially addressable.

35. The method of claim 33, wherein breaking away a portion of the breakaway seal comprises tilting and orienting the planar substrate in a direction such that the retained fluid will drain through the gap and off the planar substrate.

36. The method of claim 33, wherein the gap is created in the portion of the breakaway seal facing a perimeter edge of the planar substrate, such that the fluid is released in a direction of the facing perimeter edge off the planar substrate.

37. The method of claim 33, wherein breaking away a portion of the seal comprises:
holding the planar substrate at an incline angle, the incline angle being such that the fluid is prevented from exceeding the breakaway seal until the gap is created;
orienting the planar substrate such that a perimeter edge of the planar substrate is at a lowest position when inclined, the lowest position of the substrate perimeter edge being relative to other perimeter edges of the planar substrate; and
applying localized pressure to a sidewall of the portion of the breakaway seal surrounding the microarray, the gap being created in the sidewall, the fluid exiting through the gap off the planar substrate in the direction of the lowest substrate perimeter edge.

38. The method of claim 37, wherein the incline angle assists the fluid to flow through the gap when created.

39. The method of claim 33, further comprising:
covering the microarray with a removable cover, the cover being in contact with the breakaway seal, the cover shielding the microarray; and
removing the cover before processing the microarray, the removed cover providing fluid access to the microarray.

40. The method of claim 39, wherein the microarray is from a plurality of microarrays attached to the substrate surface in an array pattern, the cover being selectively removable from the breakaway seal, such that during removing, a section of the removable cover over the microarray is selectively removed before processing, a remainder of the removable cover being intact over the other microarrays, wherein selectively removing the section of the cover provides fluid access to the uncovered microarray while shielding the other microarrays from the fluid.

41. The method of claim 39, further comprising applying the removed cover on the fluid to help shield the fluid during processing.

42. The method of claim 33, further comprising one or more of:
rinsing the microarray with a wash solution that drains through the created gap;
scanning the microarray using scanning equipment to determine results of the processing;
storing the planar substrate until another microarray on the substrate having an intact breakaway seal is to be processed; and
processing another microarray on the substrate surrounded by an intact portion of the breakaway seal with another fluid when the processing of the microarray having the created gap in the breakaway seal is complete.

43. A removable cover for a chemical array apparatus comprising:
a sheet of material that overlies a microarray of the chemical array apparatus, the sheet being removable to provide fluid access to the microarray.

44. The removable cover of claim 43, wherein when the sheet is removed, the microarray is exposed, the removed sheet being reapplied on a fluid deposited on the exposed microarray during an assay, the reapplied sheet helping to shield the fluid during the assay of the microarray.

45. The removable cover of claim 43, wherein the chemical array apparatus comprises an array pattern of microarrays, the sheet overlying the microarrays of the array pattern, the sheet being independently removable from over a respective microarray of the array pattern, such that other microarrays of the chemical array apparatus remain covered.

46. The removable cover of claim 43, wherein the sheet of material is taut over the microarray to reduce contact between the sheet and the microarray.

47. The removable cover of claim 43, wherein the sheet is removable by peeling the sheet away from the chemical array apparatus.

48. The removable cover of claim 45, wherein the sheet is independently removably from the chemical array apparatus in sections, the independently removable sheet comprises a scored pattern corresponding to the array pattern of microarrays, such that a section of the sheet is selectively removed by separating the section along respective scoring of the scored pattern.

49. The removable cover of claim 45, wherein the sheet of material comprises a film layer and a grid frame layer, the grid frame layer having a plurality of grid frame units arranged in a frame grid pattern, the frame grid pattern corresponding to the array pattern of the microarrays, the grid frame layer being adjacent and securely attached to the chemical array apparatus, the film layer overlying the grid frame layer, the film layer being readily separable from the grid frame layer relative to the secure attachment of the grid frame layer to the chemical array apparatus.

50. The removable cover of claim 49, wherein the film layer comprises scoring in a scored pattern, the scored pattern corresponding to the grid frame pattern, the film layer being selectively separable from the grid frame layer in sections along the scoring, such that when a section of the film layer is selectively removed, the section is peeled from a respective grid frame unit and separated from a remainder of the film layer along a portion of the scoring, a remainder of the film layer being unaffected by the removal of the film layer section.
